(19) United States
(12) Patent Application Publication
(10) Pub. No.: US 2006/0084685 A1
(43) Pub. Date: Apr. 20, 2006

(54) Ophthalmological use of roflumilast for the treatment of diseases of the eye

(75) Inventors: Ruediger Koenen, Konstanz (DE); Rudolf Linder, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/515,896

(22) PCT Filed: May 27, 2003
(86) PCT No.: PCT/EP03/05536

§ 371 Date: Aug. 15, 2005

(30) Foreign Application Priority Data
May 28, 2002 (EP) 02011830.3
May 28, 2002 (DE) 102 23 828.6
Mar. 14, 2003 (DE) 103 11 613.3

(51) Int. Cl. A61K 31/44

(52) U.S. Cl. 514/352

(57) ABSTRACT

A pharmaceutical preparation comprising roflumilast for treatment of a disease of the eye is described.

### TECHNICAL FIELD

The present invention relates to a pharmaceutical preparation for treatment of diseases of the eye comprising a PDE 4 inhibitor, to processes for producing the pharmaceutical preparation and methods for treatment of diseases of the eye.

1. Prior Art

Cyclic nucleotide phosphodiesterase (PDE) inhibitors (specifically of type 4) are currently of special interest as a new generation of active ingredients for treating inflammatory disorders, especially disorders of the airways such as asthma or airway obstructions (such as, for example, COPD=chronic obstructive pulmonary disease). A number of PDE 4 inhibitors are currently undergoing advanced clinical testing, including a dosage form for oral administration comprising the active ingredient N-(3,5-dichloropyrid-4-yl)-3-cyclopropylmethoxy-4-difluoromethoxybenzamide (INN: roflumilast). This and other compounds with a benzamide structure and their use as cyclic nucleotide phosphodiesterase (PDE) inhibitors for the treatment of diseases are described in WO 95/01338.

2. Description of the Invention

Surprisingly it has now been found out, that pharmaceutical preparations comprising the PDE 4 inhibitor roflumilast show a very good effect and other beneficial properties in the treatment of diseases of the eye.

In one aspect the present invention is therefore related to the use of a compound selected from the group consisting of roflumilast, salts of roflumilast, the N-oxide of the pyridine residue of roflumilast or salts thereof for the manufacture of a pharmaceutical preparation for the prevention or treatment of a disease of the eye.

Roflumilast is the INN for a compound of the formula I 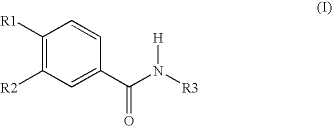
in which
R1 is difluoromethoxy,
R2 is cydopropylmethoxy and
R3 is 3,5-dichloropyrid-4-yl.

This compound has the chemical name N-(3,5-dichloropyrid-4-yl)-3-cyclopropylmethoxy-4-difluoro-methoxybenzamide (INN: roflumilast). The N-oxide of roflumilast has the chemical name 3-cyclopro-pylmethoxy-4-difluoromethoxy-N-(3,5-dichloropyrid-4-yl 1-oxide)benzamide.

This compound of the formula I, its salts, the N-oxide, its salts and the use of these compounds as phosphodiesterase (PDE) 4 inhibitors are described in the international patent application WO 95/01338.

Salts suitable for compounds of the formula I—depending on the substitution—are all acid addition salts but, in particular, all salts with bases. Particular mention may be made of the pharmacologically acceptable salts of the inorganic and organic acids and bases normally used in pharmaceutical technology. Pharmacologically unacceptable salts, which for example, may be the initial products of the process for preparing the compounds of the invention on the industrial scale are converted into pharmacologically acceptable salts by processes known to the skilled worker. Those suitable on the one hand are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid, or 3-hydroxy-2-naphthoic acid, the acids being employed to prepare the salts in the equimolar ratio of amounts, or one differing therefrom—depending on whether the acid is monobasic or polybasic and depending on which salt is desired.

On the other hand, salts with bases are also particularly suitable. Examples of basic salts which may be mentioned are lithium, sodium, potassium, calcium, aluminium, magnesium, titanium, ammonium, meglumine or guanidinium salts, once again the bases being employed to prepare the salts in the equimolar ratio of amounts or one differing therefrom.

The pharmaceutical preparations comprising roflumilast, salts of roflumilast, the N-oxide of the pyridine residue of roflumilast or salts thereof for treatment of diseases of the eye can be prepared by processes, which are known per se and familiar to the person skilled in the art. As pharmaceutical preparations, the active ingredient according to the invention can be either employed as such, or preferably in combination with suitable pharmaceutical auxiliaries, e.g. In the form of tablets, coated tablets, capsules, suppositories, patches, emulsions, suspensions, gels or solutions, the active compound content advantageously being between 0.1 and 95%. The person skilled in the art is familiar with auxiliaries, which are suitable for the desired pharmaceutical preparations on account of his expert knowledge. In addition to solvents, gel formers, ointment bases and other active compound excipients, for example antioxidants, dispersants, emulsifiers, preservatives, solubilizers or permeation promoters, can be used. Further examples, which may be mentioned, are carriers and/or excipients which are suitable for producing tablets, emulsions, suspensions, sprays, oils, ointments, greasy ointments, creams, pastes, gels, foams or solutions, and transdermal therapeutic systems.

In a preferred embodiment according to the invention the pharmaceutical preparation for treatment of diseases of the eye is an ophthalmological pharmaceutical preparation suitable for administration in, on or close to the eye.

In another embodiment the pharmaceutical preparation for treatment of diseases of the eye according to the invention is an administration form for systemic application.

Another subject of the invention is therefore an ophthalmological pharmaceutical preparation comprising a therapeutically effective and pharmacologically suitable amount of an active pharmaceutical ingredient selected from the group of compounds roflumilast, salts of roflumilast, the N-oxide of roflumilast and salts together with one or more pharmaceutically acceptable carriers and/or excipients.

Examples, which may be mentioned in connection with ophthalmological pharmaceutical preparations are eyebaths or eye lotions, eye inserts, eye ointments, eye sprays, eye drops, preparations for intraocular application [e.g. intravitreale application, intraocular injection] and eyelid ointments.

In one embodiment of the invention the ophthalmological pharmaceutical preparation is a topical pharmaceutical preparation, suitable for administration on or close to the eye comprising a therapeutically effective and pharmacologically suitable amount of an active pharmaceutical ingredient selected from the group of compounds roflumilast, salts of roflumilast, the N-oxide of roflumilast and salts together with one or more pharmaceutically acceptable carriers and/or excipients.

In another embodiment of the invention the ophthalomological pharmaceutical preparation is a pharmaceutical preparation, suitable for intravitreal and/or intraocular application comprising a therapeutically effective and pharmacologically suitable amount of an active pharmaceutical ingredient selected from the group of compounds roflumilast, salts of roflumilast, the N-oxide of roflumilast and salts together with one or more pharmaceutically acceptable carriers and/or excipients.

In a preferred embodiment of the invention the ophthalmological pharmaceutical preparation is suitable for conjunctival or palpebral administration.

In a preferred embodiment, the dosage form of the invention is an eye ointment or eye drops. Eye drops preferably comprise according to the invention aqueous or oily suspensions of the active ingredient. It is preferred in this connection for the particle size of the active ingredient employed to be 90% less than 10 μm.

Preferably used in the case of aqueous suspensions are suspension stabilizers such as, for example, substituted celluloses (e.g. methylcellulose, hydroxypropylmethylcellulose), polyvinyl alcohol, polyvinyl-pyrrolidone, in addition to preservatives (e.g. chlorocresol, phenylmercury compounds, phenylethanol, benzalkonium chloride or mixtures of individual components) and, where appropriate, sodium chloride to adjust to isotonicity. Preferably employed according to the invention in the case of oily eye drops are castor oil, peanut oil or medium chain length triglycerides. It is possible in the case of eye ointments to use according to the invention ointment bases which have the following properties: sterility or extremely low microbe content, non-irritating, good activity, good distribution of the active ingredient or its solution in the ointment, suppleness, rapid dispersion as fine film over the eyeball, good adhesion to the eye, good stability and low impairment of vision. Hydrocarbon- or cholesterol-containing bases will therefore preferably be employed according to the invention for eye ointments. In the case of petrolatum, liquid paraffin is preferably added for consistency reasons. To achieve good spreading, it is preferred according to the invention to provide compositions of limited viscosity. The viscosity at 32° C. is preferably below 1 000 mPa.s, and the yield point is preferably below 300 mPa. In the case of suspension ointments it is preferred according to the invention for 90% of the active Ingredient particles to be below 10 μm, and no particles above 90 μm should occur. In the case of water/oil emulsion ointments, it is preferred according to the invention to add preservatives such as benzalkonium chloride, thiomersal or phenylethyl alcohol.

The pharmaceutical preparation of the invention for systemic application can be a transdermal therapeutic system (TTS), for example a system as described in Pharmazeutische Technologie: Moderne Arzneiformen, Wissenschaffliche Verlagsgesellschaft mbH Stuttgart 1997, pages 81 et sec. TTSs are characterized in principle by a defined supply of medicinal substance to the skin, a total dose of the medicinal substance in the TTS, a total area and an area which is possibly different therefrom for release of the medicinal substance, a covering sheet (backing layer) which is impermeable to the medicinal substance, a medicinal substance reservoir, a control element which controls the supply of medicinal substance to the skin, a (pressure-sensitive) adhesive layer and a detachable protective layer. It is possible on occasions for more than one function to be fulfilled by one and the same element, e.g. reservoir, control and adhesive functions by a suitable adhesive matrix. From the viewpoint of pharmaceutical technology, TTSs are categorized according to the way the control function is achieved, that is to say how it controls the supply of medicinal substance to the skin. Examples, which are mentioned here are TTSs with membrane permeation-controlled release (membrane moderated drug delivery), TTSs with matrix diffusion-controlled release and TTSs with microreservoir solution-controlled release. TTSs with membrane permeation-controlled release are characterized by a polymer membrane composed of a PVA-VA copolymer (Chronomer®)) which controls the permeation of the medicinal substance from the reservoir into the skin. The medicinal substance is initially in the form of solid particles or as a dispersion or solution in the reservoir. The polymer membrane can be attached to the reservoir in various ways (extrusion, encapsulation, microencapsulation). TTSs with matrix diffusion-controlled release have a comparatively simpler structure. They contain no separate control element. The release of medicinal substance is controlled by a lipophilic or hydrophilic polymer matrix and/or the adhesive layer. It is possible to distinguish, according to the characteristics of the matrix, between TTSs with a matrix in gel form and TTSs which represent solid polymer laminates. The medicinal substance reservoir is formed by the medicinal substance dissolved in the matrix (monolithic system) or a homogeneous dispersion of solid medicinal substance particles. A matrix TTS can be produced by mixing the medicinal substance particles with a viscous liquid or semisolid polymer at room temperature, followed by crosslinking the polymer chains. A further possibility is also to mix the medicinal substance at elevated temperature with softened polymer (hot melt technique), or the two components (dissolved in an organic solvent) are mixed together and the solvent is then removed in vacuo (solvent evaporation). Shaping is possible by pouring into suitable moulds, spreading with special devices (knives) or by extrusion. In the case of TTSs with microreservoir solution-controlled release (microsealed drug delivery, MDD principle), numerous microcompartments containing the active ingredient and 10-200 μm in size are embedded in a matrix which represents both reservoir and delivery-control element. Because of the matrix, these TTSs are actually assigned to the matrix systems. For production, the medicinal substance is initially dispersed together with water and 40% polyethylene glycol 400 in isopropyl palmitate, which acts as permeation promoter. The resulting dispersion is incorporated by using a special high-energy dispersion technique into a viscous silicone elastomer which simultaneously undergoes catalytic polymerization. The medicinal substance-containing matrix can be shaped specifically by melt or extrusion techniques before it is combined with the carrier in the manner already described. Depending on the physicochemical properties of the medicinal substances and the intended liberation, it is possible to cover the matrix with a layer of a biocompatible polymer in order thus to modify the mechanism and the rate of liberation.

In another embodiment of the invention the pharmaceutical preparation for systemic administration is a dosage form for oral administration, preferably a tablet.

Suitable pharmaceutical excipients, which may be used in the dosage form for oral administration of the invention are pharmaceutical excipients such as fillers, additional binders, tablet disintegrants or else lubricants and release agents. Other suitable excipients, which may be present are, for example, flavoring substances (such as flavors and sweeteners), buffer substances, preservatives, coloring substances (such as iron oxid yellow or red) or else emulsifiers. Flavors are usually added in a proportion of from 0.05 to 1% by weight. Other flavoring substances by way of example are acids such as citric acid, sweeteners such as saccharin, aspartame, cyclamate sodium or maltol, which are added according to the desired result.

In a preferred embodiment of the invention the tablet for oral administration is employing polyvinylpyrrolidone (PVP) as binder. The polyvinylpyrrolidone (PVP) employed according to the invention is, in particular, a water-soluble PVP with an average molecular weight above 2 000, preferably above 20 000. Examples, which may be mentioned are Kollidon 12 PF (molecular weight 2 000-3 000), Kollidon 17 PF (molecular weight 7 000-11 000), Kollidon 25 (molecular weight 28 000-34 000), Kollidon 30 (molecular weight 44 000-54 000), Kollidon 90 F (molecular weight 1 000 000-1 500 000). PVP of higher molecular weight such as, for example, Kollidon 25, Kollidon 30 and Kollidon 90 F may be mentioned as preferred.

It is possible if desired to employ in addition to PVP other binders such as polyvinyl acetate (e.g. Kollidon® VA 64), gelatin, corn starch mucilage, preswollen starches (Starch 1500), hydroxypropyl-methylcellulose (HPMC) or hydroxypropylcellulose (L-HPC).

Fillers suitable according to the invention are fillers such as calcium carbonate (e.g. MagGran® CC or Destab® 95) and sodium carbonate, sugar alcohols such as mannitol (e.g. Perlitol® or Parteck® M), sorbitol (e.g. Karion®), xylitol or maltitol, starches such as corn starch, potato starch and wheat starch, microcrystalline cellulose, saccharides such as glucose, lactose (e.g. lactose monohydrate), levulose, sucrose and dextrose. It is also possible if desired to use mixtures thereof. Corn starch, microcrystalline cellulose and lactose may be mentioned as preferred.

Examples of suitable lubricants and release agents, which may be mentioned are sodium stearyl fumarate, magnesium stearate, calcium stearate, stearic acid, talc and colloidal anhydrous silica (Aerosil).

Disintegrants suitable according to the invention are, in particular, insoluble polyvinylpyrrolidone (insoluble PVP, crospovidone), carboxymethylstarch sodium [=sodium starch glycolate], sodium carboxymethylcellulose, alginic acid, and starches able to carry out the function of a disintegrant (e.g. Starch 1500).

The proportion (in percent by weight based on the finished dosage form) of PDE 4 inhibitor in the dosage form of the invention is usually, depending on the nature of the PDE 4 inhibitor, from 0.01 to 50% by weight. The proportion of PDE 4 inhibitor is preferably up to 20% by weight.

The proportion (in percent by weight based on the finished dosage form) of binder (PVP and, where appropriate, other binders) may preferably be according to the invention from 0.5 to 20% by weight. The proportion of PVP is preferably from 1 to 5% by weight, particularly preferably 2 to 3% by weight.

The proportion (in percent by weight based on the finished dosage form) of filler in the tablet of the invention is advantageously from 40 to 99% by weight. The proportion of filler is preferably from 60 to 97% by weight.

The proportion (in percent by weight based on the finished dosage form) of disintegrant in the rapidly disintegrating tablet can usually be up to 35% by weight. The proportion of disintegrant is preferably from 2 to 20% by weight. The proportion of disintegrant is particularly preferably from 5 to 10% by weight.

The proportion (in percent by weight based on the finished dosage form) of lubricant or release agent in the rapidly disintegrating tablet is usually from 0.1 to 5% by weight. The proportion of lubricant or release agent Is preferably from 0.3 to 3% by weight. The proportion of lubricant or release agent is particularly preferably from 0.5 to 2% by weight.

In a preferred embodiment of the invention, the dosage form is a tablet. It is preferred for the tablet, besides the active ingredient and PVP, to comprise as further pharmaceutical excipients at least one filler and at least one lubricant or release agent.

The pharmaceutical preparation of the invention can be produced by processes for producing tablets and pellets, which are known to the skilled worker.

In one embodiment of the invention, the pharmaceutical preparation of the invention is produced by producing a solid solution of the active ingredient in the binder PVP as carrier. This can take place for example by the solvent method in which PVP, the active ingredient and, where appropriate, other pharmaceutical excipients are dissolved in a suitable solvent, and the solvent is subsequently removed again by spray drying, normal drying, vacuum drying or freeze-drying. It has been found, surprisingly, that production of the solid solution is also possible by the mixing method in which the active ingredient and, where appropriate, other pharmaceutical excipients are vigorously mixed together with PVP.

In the event of further processing of a solid solution to tablets or pellets, the solid solution may be processed as active ingredient component together with the filler, binder, disintegrant and lubricant components by production processes familiar to the skilled worker to give the oral dosage forms of the invention. A solid solution of the active ingredient in the binder PVP as carrier means according to the invention a solid solution with amorphous structure in which the active ingredient is in the form of a molecular dispersion in the carrier material.

The pharmaceutical preparation can be produced by a process for producing a dosage form in tablet or pellet form for oral administration of the active ingredient, comprising the steps: (a) production of an active ingredient preparation in the form of a solid solution in PVP of the active ingredient,

(b) production of a mixture of an active ingredient preparation and pharmaceutical excipients and (c) granulation of the mixture obtained in (b) with an aqueous solution of PVP.

In the case of dosage forms of the invention in the form of tablets, the granules obtained in (c) can, after drying and mixing with lubricants or release agents, be compressed in a tablet press. In the case of dosage forms of the invention in the form of pellets, the wet granules obtained in (c) can be processed by the extruder/spheroidizer process to suitable pellets. Alternatively, dispersions/suspensions of an active ingredient preparation can be applied in the form of a solid solution in PVP of the active ingredient in a suitable solvent to pellet-like carriers (e.g. nonpareils or HPMC-containing pellets).

The dosage form of the invention can also be produced by granulating a mixture of active ingredient and pharmaceutical excipients with an aqueous PVP solution, drying the granules and, if desired, admixing other pharmaceutical excipients. Wet preparations obtained after granulation can then be further processed to pellets and can subsequently be packed into capsules. Dried granules can—if desired after admixture of other pharmaceutical excipients—after mixing with a release agent be compressed in a tablet press. The granulation preferably takes place in a fluidized bed granulator under suitable conditions. It is moreover possible if desired for the active ingredient to be admixed to the other pharmaceutical excipients in the form of a trituration with a pharmaceutical excipient (especially a filler). This is particularly preferred when the active ingredient content in the dosage form is less than 5% by weight. Such a trituration can normally be obtained by grinding the active ingredient with a pharmaceutical excipient (especially a filler).

The pharmaceutical preparation can therefore also be produced by a process for producing a dosage form in tablet or pellet form for oral administration of the active ingredient comprising the steps:

(a) production of a mixture of active ingredient and pharmaceutical excipients and

(b) granulation of the mixture obtained in (a) with an aqueous solution of PVP.

The pharmaceutical preparation can also be produced by granulation of a mixture of

(a) active ingredient, or a trituration of the active ingredient with corn starch,

(b) corn starch and

(c) lactose monohydrate

with an aqueous PVP solution, drying of the granules, mixing of the granules with a release agent and compression in a tablet press.

Alternatively, the pharmaceutical preparation can be produced by granulation of a mixture of

(a) active ingredient, or a trituration of the active ingredient with corn starch,

(b) corn starch,

(c) microcrystalline cellulose and

(d) sodium carboxymethylstarch

with an aqueous PVP solution, drying of the granules, mixing of the granules with a release agent and compression in a tablet press.

The pharmaceutical preparation can be produced by granulation of a mixture of pharmaceutical excipients with a suspension of the active ingredient in an aqueous PVP solution, drying of the granules and, if desired, admixture of further pharmaceutical excipients. The preparations obtained in this way can then, after mixing with a release agent, be compressed in a tablet press. The granulation preferably takes place in a fluidized bed granulator under suitable conditions.

The pharmaceutical preparation can also be produced by a process comprising the steps:

(a) production of a mixture of pharmaceutical excipients and

(b) granulation of the mixture obtained in (a) with a suspension of the active ingredient in an aqueous solution of PVP.

The pharmaceutical preparation can be produced by granulation of a mixture of corn starch and lactose monohydrate with a suspension of the active ingredient in an aqueous solution of PVP, drying of the granules, mixing of the granules with a release agent and compression in a tablet press.

The production of pharmaceutical preparation according to the invention is described by way of example below. The following examples explain the invention in more detail without restricting it.

### EXAMPLES

Production of Pharmaceutical Preparations of the Invention

### Example 1

Composition of an Eye Ointment (Quantity for 1 000 Grams)

| | | |
|---|---|---|
| Roflumilast | 1 | g |
| Cetyl alcohol | 4 | g |
| High-viscosity paraffin | 200 | g |
| White petrolatum | 795 | g |

Production: A clear melt of the cetyl alcohol, the high-viscosity paraffin and the white petrolatum is prepared at about 70° C. The micronized roflumilast (90% of the particles below 10 μm) is stirred in, and a homogeneous dispersion is prepared using an Ultra-Turrax. The suspension is cooled to room temperature while stirring and used to fill suitable tubes.

### Example 2

Composition of a Drop Solution in the Form of an Emulsion (Quantity for 1 000 Millilitres)

| | | |
|---|---|---|
| Roflumilast | 1.5 | g |
| Medium chain length triglycerides | 100.0 | g |
| Lecithin | 12.0 | g |
| Glycerol | 25.0 | g |
| Thiomersal | 0.1 | g |
| Purified water | to 1000 | ml |

Production: First the roflumilast and then the lecithin are dissolved in the medium chain length triglycerides and the glycerol at 30° C.-40° C. While stirring vigorously, the purified water is added and then homogenized until the droplet size of the disperse phase is below 500 nm. The thiomersal is dissolved by stirring. The emulsion is filtered through a 0.45 μm filter and dispensed into suitable containers.

### Example 3

Composition for a Drop Solution in Form of an Emulsion (Quantity for 1000 Millilitres)

| | | |
|---|---|---|
| Roflumilast | 1.5 | g |
| Lecithin | 1.5 | g |
| Thiomersal | 0.1 | g |
| Polyvidone (Kollidon ® 17) | 10.0 | g |
| 0.9% sodiumchloride solution | to 1000 | ml |

While stirring vigorously the lecithin, thiomersal and polyvidone are dissolved in the 0,9% sodiumchloride solution. The micronized Roflumilast (90% of particles below 10 μm) is stirred in and homogeneously dispersed.

Production of Tablets of the Invention

### Example A

Weight Based on a Tablet Containing 0.1 mg of Roflumilast

| | | | |
|---|---|---|---|
| 1. | Roflumilast (micronized) | 0.100 | mg |
| 2. | Lactose monohydrate | 49.660 | mg |
| 3. | Corn starch | 13.390 | mg |
| 4. | Polyvidone K90 | 1.300 | mg |
| 5. | Magnesium stearate (vegetable) | 0.650 | mg |
| | Total | 65.100 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 65.1 mg.

### Example B

Weight Based on a Tablet Containing 0.125 mg of Roflumilast

| | | | |
|---|---|---|---|
| 1. | Roflumilast | 0.125 | mg |
| 2. | Lactose monohydrate | 49.660 | mg |
| 3. | Corn starch | 13.390 | mg |
| 4. | Polyvidone K90 | 1.300 | mg |
| 5. | Magnesium stearate (vegetable) | 0.650 | mg |
| | Total | 65.125 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 65.125 mg.

### Example C

Weight Based on a Tablet Containing 0.25 mg of Roflumilast

| | | | |
|---|---|---|---|
| 1. | Roflumilast | 0.250 | mg |
| 2. | Microcrystalline cellulose | 33.900 | mg |
| 3. | Corn starch | 2.500 | mg |
| 4. | Polyvidone K90 | 2.250 | mg |
| 5. | Sodium carboxymethylstarch (type A) | 20.000 | mg |
| 6. | Magnesium stearate (vegetable) | 0.600 | mg |
| | Total | 59.500 | mg |

Production: (1) is mixed with part of (3), and a trituration is produced in a planetary mill. The trituration is put together with (2), (5) and the remaining amount of (3) in the product container of a fluidized bed granulation system, and a 5% granulation solution of (4) in purified water is sprayed on and dried under suitable conditions. (6) is added to the granules, and the mixture obtained after mixing is compressed In a tablet press to tablets having an average weight of 59.5 mg.

### Example D

Weight Based on a Tablet Containing 0.25 mg of Roflumilast

| | | | |
|---|---|---|---|
| 1. | Roflumilast | 0.250 | mg |
| 2. | Lactose monohydrate | 49.660 | mg |
| 3. | Corn starch | 13.390 | mg |
| 4. | Polyvidone K90 | 1.300 | mg |
| 5. | Magnesium stearate (vegetable) | 0.650 | mg |
| | Total | 65.250 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 65.25 mg.

### Example E

Weight Based on a Tablet Containing 0.5 mg of Roflumilast

| | | | |
|---|---|---|---|
| 1. | Roflumilast | 0.500 | mg |
| 2. | Lactose monohydrate | 49.660 | mg |
| 3. | Corn starch | 13.390 | mg |
| 4. | Polyvidone K90 | 1.300 | mg |
| 5. | Magnesium stearate (vegetable) | 0.650 | mg |
| | Total | 65.500 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 65.500 mg.

### Example F

Weight Based on a Tablet Containing 0.5 mg of Roflumilast

| | | | |
|---|---|---|---|
| 1. | Roflumilast | 0.500 | mg |
| 2. | Lactose monohydrate | 99.320 | mg |
| 3. | Corn starch | 26.780 | mg |
| 4. | Polyvidone K90 | 2.600 | mg |
| 5. | Magnesium stearate (vegetable) | 1.300 | mg |
| | Total | 130.500 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 130.5 mg.

### Example G

Weight Based on a Tablet Containing 2.5 mg of Roflumilast

| | | | |
|---|---|---|---|
| 1. | Roflumilast | 2.500 | mg |
| 2. | Microcrystalline cellulose | 33.900 | mg |
| 3. | Corn starch | 2.500 | mg |
| 4. | Polyvidone K90 | 2.250 | mg |
| 5. | Sodium carboxymethylstarch (type A) | 20.000 | mg |
| 6. | Magnesium stearate (vegetable) | 0.600 | mg |
| | Total | 61.750 | mg |

Production: (1) is mixed with part of (3), and a trituration is produced in a planetary mill. The trituration is put together with (2), (5) and the remaining amount of (3) in the product container of a fluidized bed granulation system, and a 5% granulation solution of (4) in purified water is sprayed on and dried under suitable conditions. (6) is added to the granules, and the mixture obtained after mixing is compressed in a tablet press to tablets having an average weight of 61.75 mg.

### Example H

Production of Tablets Containing 0.1 mg of Roflumilast as Active Ingredient (Weight for a Batch of 70 000 tablets)

| | | | |
|---|---|---|---|
| 1. | Roflumilast (micronized) | 7.000 | g |
| 2. | Lactose monohydrate | 3476.200 | g |
| 3. | Corn starch | 937.300 | g |
| 4. | Polyvidone K90 | 91.000 | g |
| 5. | Magnesium stearate (vegetable) | 45.500 | g |
| | Total | 4557.000 | g |

Production: (1) is mixed with 70 g of (3), and a trituration is produced in a planetary mill. The trituration is put together with (2) and the remaining amount of (3) in the product container of a fluidized bed granulation system, and a 5% granulation solution of (4) in purified water is sprayed on. (Spraying pressure: 3 bar; product temperature: 28-33° C.; air flow rate in the first third of the spraying process: 100 m³/h; air flow rate subsequently during the spraying process: 150 m³/h; inlet air temperature: 40-70° C.; spraying rate: 30-40 g/min). After spraying is complete, drying is carried out until the product temperature reaches 34° C. The granules are passed through a stainless steel sieve with a mesh width of 0.8 mm, and the relative surface moisture is measured and adjusted to a value in the range 20-50%. (5) is added to the granules, and the mixture obtained after mixing is compressed in a tablet press to tablets having an average weight of 65.1 mg.

### Example I

Production of Tablets Containing 0.25 mg of Roflumilast as Active Ingredient (Weight for a Batch of 70 000 tablets)

| | | | |
|---|---|---|---|
| 1. | Roflumilast (micronized) | 35.000 | g |
| 2. | Lactose monohydrate | 3476.200 | g |
| 3. | Corn starch | 937.300 | g |
| 4. | Polyvidone K90 | 91.000 | g |
| 5. | Magnesium stearate (vegetable) | 45.500 | g |
| | Total | 4585.000 | g |

Production: 19.25 g of (1) are mixed with 192.5 g of (3), and a trituration is produced in a planetary mill. The trituration is put together with (2) and the remaining amount of (3) in the product container of a fluidized bed granulation system, and a 5% granulation solution of (4) in purified water is sprayed on. (Spraying pressure: 3 bar; product temperature: 28-33° C.; air flow rate in the first third of the spraying process: 100 m³/h; air flow rate subsequently during the spraying process: 150 m³/h; inlet air temperature: 40-70° C.; spraying rate: 30-40 g/min). After spraying is complete, drying is carried out until the product temperature reaches 34° C. The granules are passed through a stainless steel sieve with a mesh width of 0.8 mm, and the relative surface moisture is measured and adjusted to a value in the range 20-50%. (5) is added to the granules, and the mixture obtained after mixing is compressed in a tablet press to tablets having an average weight of 65.5 mg.

### Example J

Production of Tablets Containing 0.1 mg of Roflumilast as Active Ingredient (Weight for a Batch of 70 000 tablets)

| | | | |
|---|---|---|---|
| 1. | Roflumilast (micronized) | 7.000 | g |
| 2. | Lactose monohydrate | 3476.200 | g |
| 3. | Corn starch | 937.300 | g |
| 4. | Polyvidone K90 | 91.000 | g |
| 5. | Magnesium stearate (vegetable) | 45.500 | g |
| | Total | 4557.000 | g |

Production: (1) is homogeneously suspended in a granulation solution of (4) in purified water. (2) and (3) are put into the product container of a suitable fluidized bed granulation system and granulated with the granulation suspension described above, and then dried. (5) is added to the granules, and the mixture obtained after mixing is compressed in a tablet press to tablets having an average weight of 65.1 mg.

### Example K

Production of Tablets Containing 0.25 mg of Roflumilast as Active Ingredient (Weight for a Batch of 70 000 Tablets)

| | | | |
|---|---|---|---|
| 1. | Roflumilast (micronized) | 35.000 | g |
| 2. | Lactose monohydrate | 3476.200 | g |
| 3. | Corn starch | 937.300 | g |
| 4. | Polyvidone K90 | 91.000 | g |
| 5. | Magnesium stearate (vegetable) | 45.500 | g |
| | Total | 4585.000 | g |

Production: (1) is homogeneously suspended in a granulation solution of (4) in purified water. (2) and (3) are put into the product container of a suitable fluidized bed granulation system and granulated with the granulation suspension described above, and then dried. (5) is added to the granules, and the mixture obtained after mixing is compressed in a tablet press to tablets having an average weight of 65.25 mg.

### Example L

Weight Based on a Tablet Containing 0.25 mg of Roflumilast

| | | | |
|---|---|---|---|
| 1. | Roflumilast | 0.250 | mg |
| 2. | Lactose monohydrate | 49.660 | mg |
| 3. | Potato starch | 10.000 | mg |
| 4. | Corn starch | 3.590 | mg |
| 5. | PVP 25 | 1.500 | mg |
| 6. | Magnesium stearate (vegetable) | 0.650 | mg |
| | Total | 65.650 | mg |

Production: A dispersion is produced from (4) and water, and (1) is homogeneously suspended therein. (5) is dissolved in water and added to the dispersion. (2) and (3) are granulated in a suitable fluidized bed granulation system with the dispersion under suitable conditions. (6) is added to this mixture, and the mixture obtained after mixing is compressed in a tablet press to tablets having an average weight of 65.650 mg.

### Example M

Weight Based on a Tablet Containing 0.25 mg of Roflumilast

| | | | |
|---|---|---|---|
| 1. | Roflumilast | 0.250 | mg |
| 2. | Lactose monohydrate | 49.660 | mg |
| 3. | Corn starch | 13.390 | mg |
| 4. | Polyvidone K90 | 1.300 | mg |
| 5. | Gelatin | 1.300 | mg |
| 6. | Magnesium stearate (vegetable) | 0.650 | mg |
| | Total | 66.550 | mg |

Production: (1) is mixed with part of (3), and a trituration is produced in a planetary mill. The trituration is put together with (2) and the remaining amount of (3) in the product container of a fluidized bed granulation system, and a 5% granulation solution of (4) and (5) in purified water is sprayed on and dried under suitable conditions. (6) is added to the granules, and the mixture obtained after mixing is compressed in a tablet press to tablets having an average weight of 66.55 mg.

### Example M1

Formulation for Pediatric Use

Weight Based on a Tablet Containing 0.125 mg of Roflumilast

| | | | |
|---|---|---|---|
| 1. | Roflumilast | 0.125 | mg |
| 2. | Lactose monohydrate | 49.660 | mg |
| 3. | Corn starch | 13.390 | mg |
| 4. | Polyvidone K90 | 1.300 | mg |
| 5. | Mannit | 32.238 | mg |
| 6. | Flavor (Tutti Frutti) | 0.329 | mg |
| 7. | PVP (insoluble) | 12.895 | mg |
| 5. | Magnesium stearate (vegetable) | 1.649 | mg |
| | Total | 111.586 | mg |

The formulation is produced according to a process disclosed above.

### INDUSTRIAL APPLICABILITY

The pharmaceutical preparations of the invention can be employed for the treatment and prevention (prophylaxis) of all eye diseases regarded as treatable or preventable through the use of PDE4 inhibitors. The pharmaceutical preparations according to the invention are suitable for treatment of diseases of the eye selected from the group of blepharitis, not-infective dermatosis of palpebrae in general, dermatosis of palpebrae in general (allergic, eczematous, contact), erythematodes chronicus discoides, chalazion, chronical inflammations of palpebrae, epiphora, chronical dakryocystitis, canaliculitis, conjunctivitis (allergic, acute), keratoconjunctivitis, chronic conjunctivitis, blepharoconjuncivitis, conjunctival hyperaemia, conjunctival oedema, pseudopterygium, ocular or conjunctival pemphigus, episkleritis, skleritis, inflammation of sclera and episclera, staphyloma, keratitis (areolar, chronic filaminatary, mummular, descemetitis, stellar, striate), photokeratitis, solar- and photoophtalmia, keratoconjunctivitis (neuroparalytical, phlyctenar, by exposition), superficial keratitis with conjunctivitis, dry eye syndrome (keratitis sicca), ophtalmia, moorens ulcer, cicatrix, corneal opacitiy, interstitial and profound keratitis, corneal neovascularisation (degeneration and erosion), iridocyclitis due to sarcoidosis or Bechterew's disease, acute and chronical iridocyclitis, iritis, anterior uveitis, iris atrophy, precipitation on the cornea, inflammatory states after intraocular lens implantation, retinal oedema, (postsurgical) macula oedema, granulomatous uveitis, prevention of postsurgical, inflammatory complications, glaucoma, cataract due to chronical iridocyclitis, pigmentous retinitis or Usher's syndrom, diabetic retinopathia, macular degeneration, optic retrobulbar neuritis, neuromyelitis, vitreoretinopathy, inflammatory states after intraocular lens implantation and retinal oedema.

The invention further relates to a method for the treatment of mammals, including humans, suffering from one of the abovementioned diseases. The method is characterized in that a therapeutically effective and pharmacologically suitable amount of an active pharmaceutical ingredient selected from the group of compounds roflumilast, salts of roflumilast, the N-oxide of roflumilast and salts thereof is administered to the mammal with the disease.

In another preferred embodiment, the invention relates to the treatment of mammals, including humans, suffering from an eye disorder, which is regarded as treatable or preventable through the use of PDE4 inhibitors.

In one embodiment of the invention the method is characterized that the administration takes place by systemic or topical application of the pharmaceutical preparation. In this case the eye disorder is preferably selected from the group of blepharitis, not-infective dermatosis of palpebrae in general, dermatosis of palpebrae in general (allergic, eczematous, contact), erythematodes chronicus discoides, chalazion, chronical inflammations of palpebrae, epiphora, chronical dakryocystitis, canaliculitis, conjunctivitis (allergic, acute), keratoconjunctivitis, chronic conjunctivitis, blepharoconjuncivitis, conjunctival hyperaemia, conjunctival oedema, pseudopterygium, ocular or conjunctival pemphigus, episkleritis, skleritis, inflammation of sclera and episclera, staphyloma, keratitis (areolar, chronic filaminatary, mummular, descemetitis, stellar, striate), photokeratitis, solar- and photoophtalmia, keratoconjunctivitis (neuroparalytical, phlyctenar, by exposition), superficial keratitis with conjunctivitis, dry eye syndrome (keratitis sicca), ophtalmia, moorens ulcer, cicatrix, corneal opacitiy, interstitial and profound keratitis, corneal neovascularisation (degeneration and erosion), iridocyclitis due to sarcoldosis or Bechterew's disease, acute and chronical iridocyclitis, iritis, anterior uveitis, iris atrophy, precipitation on the cornea, inflammatory states after intraocular lens implantation, retinal oedema.

In another embodiment of the invention the method is characterized that the administration takes place by systemic or by intraocular and/or intravitreal administration of the pharmaceutical preparation. In this case the eye disorder is preferably selected from the group of (postsurgical) macula oedema, granulomatous uveitis, prevention of postsurgical, inflammatory complications, glaucoma, cataract due to chronical iridocyclitis, pigmentous retinitis or Usher's syndrom, diabetic retinopathia, macular degeneration, optic retrobulbar neuritis, neuromyelitis, vitreoretinopathy, inflammatory states after intraocular lens implantation and retinal oedema.

The pharmaceutical preparations of the invention comprise the active pharmaceutical ingredient in the dose customary for the treatment of the particular disease. The dosage of the active ingredient is of the order of magnitude customary for PDE inhibitors, it being possible to administer the daily dose in one or more dosage units. Customary dosages are disclosed for example in WO 95/01338. The normal dose on systemic therapy (oral) is between 0.001 and 3 mg per kilogram and day. Pharmaceutical preparations preferred according to the invention for topical administration contain from 0.005 mg to 5 mg of roflumilast, preferably from 0.01 mg to 2.5 mg, particularly preferably 0.1 mg to 0.5 mg of roflumilast per dosage unit. Examples of pharmaceutical preparations of the invention contain 0.01 mg, 0.1 mg, 0.125 mg, 0.25 mg and 0.5 mg of roflumilast per dosage unit.

1. Use of a compound selected from the group consisting of roflumilast, salts of roflumilast, the N-oxide of the pyridine residue of roflumilast or salts thereof for the manufacture of a pharmaceutical preparation for the prevention or treatment of a disease of the eye.

2. Use according to claim 1 wherein roflumilast is a compound of the formula I 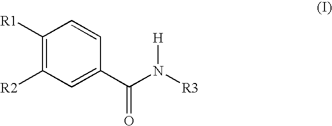
in which
R1 is difluoromethoxy,
R2 is cyclopropylmethoxy and
R3 is 3,5-dichloropyrid-4-yl.

3. Use according to claim 1, wherein the disease of the eye is selected from the group of blepharitis, not-infective dermatosis of palpebrae in general, dermatosis of palpebrae in general (allergic, eczematous, contact), erythematodes chronicus discoides, chalazion, chronical inflammations of palpebrae, epiphora, chronical dakryocystitis, canaliculitis, conjunctivitis (allergic, acute), keratoconjunctivitis, chronic conjunctivitis, blepharoconjuncivitis, conjunctival hyperaemia, conjunctival oedema, pseudopterygium, ocular or conjunctival pemphigus, episkleritis, skleritis, inflammation of sclera and episclera, staphyloma, keratitis (areolar, chronic filaminatary, mummular, descemetitis, stellar, striate), photokeratitis, solar- and photoophtalmia, keratoconjunctivitis (neuroparalytical, phlyctenar, by exposition), superficial keratits with conjunctivitis, dry eye syndrome (keratitis sicca), ophtalmia, moorens ulcer, cicatrix, corneal opacitiy, interstitial and profound keratitis, corneal neovascularisation (degeneration and erosion), iridocyclitis due to sarcoidosis or Bechterew's disease, acute and chronical iridocyclitis, iritis, anterior uveitis, iris atrophy, precipitation on the cornea, inflammatory states after intraocular lens implantation, retinal oedema, (postsurgical) macula oedema, granulomatous uveitis, prevention of postsurgical, inflammatory complications, glaucoma, cataract due to chronical iridocyclitis, pigmentous retinitis or Usher's syndrom, diabetic retinopathia, macular degeneration, optic retrobulbar neuritis, neuromyelitis, vitreoretinopathy, inflammatory states after intraocular lens implantation and retinal oedema

4. Use according to claim 1, wherein the pharmaceutical preparation is an ophthalmological pharmaceutical preparation.

5. Use according to claim 4, wherein the pharmaceutical preparation is selected from the group of eyebaths, eye lotions, eye inserts, eye ointments, eye sprays, eye drops, preparations for intraocular application and eyelid ointments.

6. Use according to claim 1, wherein the pharmaceutical preparation is suitable for systemic application.

7. Method for treating mammals, including humans, suffering from a disease of the eye regarded as treatable or preventable through use of PDE 4 inhibitors, characterized in that a therapeutically effective and pharmacologically suitable amount of an active pharmaceutical ingredient selected from the group of compounds roflumilast, salts of roflumilast, the N-oxide of roflumilast and salts thereof is administered to the mammal with the disorder.

8. Method according to claim 8, where the disease is selected from the group of blepharitis, not-infective dermatosis of palpebrae in general, dermatosis of palpebrae in general (allergic, eczematous, contact), erythematodes chronicus discoides, chalazion, chronical inflammations of palpebrae, epiphora, chronical dakryocystitis, canaliculitis, conjunctivitis (allergic, acute), keratoconjunctivitis, chronic conjunctivitis, blepharoconjuncivitis, conjunctival hyperaemia, conjunctival oedema, pseudopterygium, ocular or conjunctival pemphigus, episkleritis, skleritis, inflammation of sclera and episclera, staphyloma, keratitis (areolar, chronic filaminatary, mummular, descemetitis, stellar, striate), photokeratitis, solar- and photoophtalmia, keratoconjunctivitis (neuroparalytical, phlyctenar, by exposition), superficial keratits with conjunctivitis, dry eye syndrome (keratitis sicca), ophtalmia, moorens ulcer, cicatrix, corneal opacitiy, interstitial and profound keratitis, corneal neovascularisation (degeneration and erosion), iridocyclitis due to sarcoidosis or Bechterew's disease, acute and chronical iridocyclitis, iritis, anterior uveitis, iris atrophy, precipitation on the cornea, inflammatory states after intraocular lens implantation, retinal oedema, (postsurgical) macula oedema, granulomatous uveitis, prevention of postsurgical, Inflammatory complications, glaucoma, cataract due to chronical iridocyclitis, pigmentous retinitis or Usher's syndrom, diabetic retinopathia, macular degeneration, optic retrobulbar neuritis, neuromyelitis, vitreoretinopathy, inflammatory states after intraocular lens implantation and retinal oedema

9. Ophthalmological pharmaceutical preparation comprising a therapeutically effective and pharmacologically suitable amount of an active pharmaceutical ingredient selected from the group of compounds roflumilast, salts of roflumilast, the N-oxide of roflumilast and salts together with one or more pharmaceutically acceptable carriers and/or excipients.

10. Ophthalmological pharmaceutical preparation selected from the group of eyebaths, eye lotions, eye inserts, eye ointments, eye sprays, eye drops, preparations for intraocular application and eyelid ointments.

11. Pharmaceutical preparation according to claim 10, characterized in that it comprises a suspension of the active pharmaceutical ingredient in the carriers and/or the excipients.

12. Pharmaceutical preparation according to claim 11, which is in the form of eye drops and wherein the particle size of 90% of the active pharmaceutical ingredient is less than 10 μm.
